(19) United States
(12) Patent Application Publication
(10) Pub. No.: US 2007/0073094 A1
(43) Pub. Date: Mar. 29, 2007

(54) Process for the production of olefins

(76) Inventors: Ian Raymond Little, Naperville, IL (US); Vaughan Clifford Williams, Staines (GB)

Correspondence Address: NIXON & VANDERHYE, PC, 901 NORTH GLEBE ROAD, 11TH FLOOR, ARLINGTON, VA, 22203, US

(21) Appl. No.: 10/556,784

(22) PCT Filed: May 12, 2004
(86) PCT No.: PCT/GB04/02035

§ 371 Date: Nov. 14, 2005

(30) Foreign Application Priority Data
May 27, 2003 (GB) 0312093.8

(51) Int. Cl. C07C 5/327; C07C 5/373

(52) U.S. Cl. 585/658

(57) ABSTRACT

The present invention relates to a process for the production of an olefin, said process comprising passing a mixture of a hydrocarbon and an oxygen-containing gas through a catalyst zone which is capable of supporting combustion beyond the fuel rich limit of flammability to produce said olefin, said catalyst zone comprising at least a first catalyst bed and a second catalyst bed, and wherein the second catalyst bed is located downstream of the first catalyst bed, is of a different composition to the first catalyst bed and has the general formula of: M¹ₐM²_{b}M³_{c}O_{z} wherein M¹ is selected from groups IIA, JIB, IIIB, IVB, VB, VIIB, VIIB, lanthanides and actinides, M² is selected from groups IIA, IB, JIB, IIIB, IVB, VB, VIB, and M³ is selected from groups IIA, IB, IIB, IIIB, IVB, VB, VIB and VIIIB.

The present invention relates to a process for the production of olefins from hydrocarbons in which the hydrocarbons are treated to autothermal cracking.

Autothermal cracking is a new route to olefins in which the hydrocarbon feed is mixed with oxygen and passed over an autothermal cracking catalyst. The autothermal cracking catalyst is capable of supporting combustion beyond the fuel rich limit of flammability. Combustion is initiated on the catalyst surface and the heat required to raise the reactants to the process temperature and to carry out the endothermic cracking process is generated in situ. Generally the hydrocarbon feed and the oxygen is passed over a single catalyst bed to produce the olefin product. Typically, the catalyst bed comprises at least one platinum group metal, for example, platinum, supported on a catalyst support. The autothermal cracking process is described in EP 332289B; EP-529793B; EP-A-0709446 and WO 00/14035.

The autothermal cracking process produces a product stream that contains not only a range of paraffinic and olefinic components but also significant quantities of hydrogen and carbon monoxide. WO 02/04389 has shown that the selectivity of a catalyst zone comprising a catalyst bed (a first catalyst bed) can be enhanced by positioning a second catalyst bed comprising at least one metal selected from the group consisting of Mo, W, and Group IB, IIB, IIIB, IVB, VB, VIIB and VIII of the Periodic Table downstream of the first catalyst bed. In particular WO 02/04389 shows that the use of a catalyst zone which comprises as the second catalyst bed, a catalyst which is substantially incapable of supporting combustion beyond the fuel rich limit of flammability (that is, a catalyst which is substantially inactive under autothermal cracking conditions), and as the first catalyst bed, a catalyst which is substantially capable of supporting combustion beyond the fuel rich limit of flammability, generally achieves greater olefin selectivity compared to that obtained by the use of the first catalyst bed alone.

It has now been found that the olefin selectivity of a catalyst zone comprising a catalyst bed (a first catalyst bed) can be enhanced by positioning a second catalyst bed of formula M¹ₐM²_{b}M³_{c}O_{z}, wherein M¹ is selected from groups IIA, IB, IIIB, IVB, VB, VIIB, VIIB, lanthanides and actinides, M² is selected from groups IIA, IB, IIB, IIIB, IVB, VB, VIB, and M³ is selected from groups IIA, IB, IIB, IIIB, IVB, VB, VIB and VIIIB, downstream of said first catalyst bed.

Accordingly, the present invention provides a process for the production of an olefin, said process comprising passing a mixture of a hydrocarbon and an oxygen-containing gas through a catalyst zone which is capable of supporting combustion beyond the fuel rich limit of flammability to produce said olefin, said catalyst zone comprising at least a first catalyst bed and a second catalyst bed, and wherein the second catalyst bed is located downstream of the first catalyst bed, is of a different composition to the first catalyst bed and has the general formula of:
M¹ₐM²_{b}M³_{c}O_{z}
wherein M¹ is selected from groups IIA, IIB, IIIB, IVB, VB, VIB, VIIB, lanthanides and actinides, M² is selected from groups IIA, IB, IIB, IIIB, IVB, VB, VIB, M³ is selected from groups IIA, IB, IIB, IIIB, IVB, VB, VIIB and VIIB, a, b, c and z are the atomic ratios of components M¹, M², M³ and O respectively, a is in the range of 0.1 to 1.0, b is in the range of 0.1 to 2.0, c is in the range of 0.1-3.0, and z is in the range 0.1 to 9.

The first catalyst bed comprises a catalyst which is capable of supporting combustion beyond the fuel rich limit of flammability. Suitably, the first catalyst bed may comprise a Group VIIIB metal. Suitable Group VIIIB metals include platinum, palladium, ruthenium, rhodium, osmium and iridium. Preferably, the Group VIIIB metal is selected from rhodium, platinum, palladium or mixtures thereof. Especially preferred are platinum, palladium or mixtures thereof. Typical Group VIIIB metal loadings range from 0.01 to 100 wt %, preferably, from 0.01 to 20 wt %, and more preferably, from 0.01 to 10 wt %, for example 1-5 wt %, such as 3-5 wt %. Suitably, the first catalyst bed comprises platinum or palladium, especially platinum.

Alternatively, the first catalyst bed may comprise a promoted catalyst such as a promoted Group VIIIB metal catalyst. The promoter may be selected from the elements of Groups IIIA, IVA and VA of the Periodic Table and mixtures thereof. Alternatively, the promoter may be a transition metal; the transition metal being a different metal to the catalyst component, such as the Group VIIIB metal(s) employed as the catalytic component.

Preferred Group IIIA metals include Al, Ga, In and Ti. Of these, Ga and In are preferred. Preferred Group IVA metals include Ge, Sn and Pb. Of these, Ge and Sn are preferred, especially Sn. The preferred Group VA metal is Sb. The atomic ratio of Group VIIIB metal to the Group IIIA, IVA or VA metal may be 1:0.1-50.0, preferably, 1:0.1-12.0, such as 1:0.3-5.

Suitable transition metal promoters may be selected from any one or more of Groups IB to VIIIB of the Periodic Table. In particular, transition metals selected from Groups IB, IIB, VIB, VIIB and VIIIB of the Periodic Table are preferred. Examples of such transition metal promoters include Cr, Mo, W, Fe, Ru, Os, Co, Rh, Ir, Ni, Pt, Cu, Ag, Au, Zn, Cd and Hg. Preferred transition metal promoters are Mo, Rh, Ru, Ir, Pt, Cu and Zn, especially Cu. The atomic ratio of the Group VIIIB metal to the transition metal promoter may be 1:0.1-50.0, preferably, 1:0.1-12.0.

Specific examples of promoted Group VIIIB catalysts for use as the first catalyst bed include Pt/Ga, Pt/In, Pt/Sn, Pt/Ge, Pt/Cu, Pd/Sn, Pd/Ge, Pd/Cu and Rh/Sn. Where the Group VIIIB metal is Rh, Pt or Pd, the Rh, Pt or Pd may comprise between 0.01 and 5.0 wt %, preferably, between 0.01 and 2.0 wt %, and more preferably, between 0.05 and 1.0 wt % of the total weight of the catalyst. The atomic ratio of Rh, Pt or Pd to the Group IIIA, IVA, VA or transition metal promoter may be 1:0.1-50.0, preferably, 1:0.1-12.0. For example, atomic ratios of Rh, Pt or Pd to Sn may be 1:0.1 to 50, preferably, 1:0.1-12.0, more preferably, 1:0.2-3.0 and most preferably, 1:0.5-1.5. Atomic ratios of Pt or Pd to Ge may be 1:0.1 to 50, preferably, 1:0.1-12.0, and more preferably, 1:0.5-8.0. Atomic ratios of Pt or Pd to Cu may be 1:0.1-3.0, preferably, 1:0.2-2.0, and more preferably, 1:0.5-1.5.

The second catalyst bed generally has the formula of;
M¹ₐM²_{b}M³_{c}O_{z}
wherein M¹ is selected from groups IIA, IIB, IIIB, IVB, VB, VIB, VIIB, lanthanides and actinides, M² is selected from groups IIA, IB, IIB, IIIB, IVB, VB, VIB, and M³ is selected from groups IIA, IB, IIB, IIIB, IVB, VB, VIB and VIIIB. (As used herein the groups of the Periodic Table are referenced using the CAS notation, as listed in Advanced Inorganic Chemistry, Fifth edition, 1988, by Cotton and Wilkinson.)

Preferably M¹ is selected from group IIIB, M² is selected from group IIA and M³ is selected from group IB. Most preferably M¹ is yttrium, M² is barium and M³ is copper.

The materials shown in the formula above may be present as a mixture of the individual oxide components generally having the formula of;
M¹ₓ₁O_{y1}, M²ₓ₂O_{y2}, M³ₓ₃O_{y3}
wherein M¹, M² and M³ are as herein described above and wherein x1, x2, x3, y1, y2 and y3 are in the range of 1-7, and such that the three individual oxide components are mixed in suitable proportions to give the atomic ratios for M¹, M² and M³ of a, b and c respectively.

The second catalyst bed is preferably in the form of a perovskite. Perovskite-type structures include yttrium-barium-copper oxides YBa₂Cu₃O_{7-δ}, lanthanum-strontium-iron oxides La₁₋ₓSrₓFeO_{3-δ}, and lanthanum-manganese-copper oxides LaMn₁₋ₓCuₓO_{3-δ}, wherein x is in the range of 0.1-0.9 and δ is typically in the range of 0.01-1, preferably in the range 0.01-0.25.

The second catalyst bed may be promoted by addition of halide-promoters to yield materials of having the general formula of;
M¹ₐM²_{b}M³_{c}XₓO_{z}
wherein M¹, M² and M³ and a, b, c and z are as herein described above, X is a halide, preferably F or Cl, and x is typically in the range of 0.05-0.5.

A preferred halide-promoted second catalyst bed is YBa₂Cu_{3-δ}Cl_{σ} wherein δ is usually in the range 0.01-0.25, and σ is usually in the range of 0.05-0.3.

In addition to the first and second catalyst beds the catalyst zone may comprise further catalyst beds. For example, the catalyst zone may comprise 3 to 10, preferably, 3 to 5 catalyst beds.

Where the catalyst zone comprises more than two catalyst beds, the catalyst of the additional bed(s) may be the same or different to the catalysts used for either of the first and second catalyst beds. Suitably, the catalyst used for the additional bed(s) is the same as that of the second catalyst bed.

Each catalyst employed in the catalyst zone may be unsupported or supported. Suitably, an unsupported catalyst may be in the form of a metal gauze. Preferably, at least one catalyst in the catalyst zone is a supported catalyst. Suitably, each catalyst in the catalyst zone is a supported catalyst. The support used for each catalyst may be the same or different. Although a range of support materials may be used, ceramic supports are generally preferred. However, metal supports may also be used.

Suitably, the ceramic support may be any oxide or combination of oxides that is stable at high temperatures of, for example, between 600° C. and 1200° C. The ceramic support material preferably has a low thermal expansion co-efficient, and is resistant to phase separation at high temperatures.

Suitable ceramic supports include cordierite, lithium aluminium silicate (LAS), alumina (alpha-Al₂O₃), yttria stabilised zirconia, aluminium titanate, niascon, and calcium zirconyl phosphate, and, in particular, alumina.

The ceramic support may be wash-coated, for example, with gamma-Al₂O₃.

The structure of the support material is important, as the structure may affect flow patterns through the catalyst. Such flow patterns may influence the transport of reactants and products to and from the catalyst surface, thereby affecting the activity of the catalyst. Typically, the support material may be in the form of particles, such as spheres or other granular shapes or it may be in the form of a foam or fibre such as a fibrous pad or mat. Suitably, the particulate support material may be alumina spheres. Preferably, the form of the support is a monolith which is a continuous multi-channel ceramic structure. Such monoliths include honeycomb structures, foams, or fibrous pads. The pores of foam monolith structures tend to provide tortuous paths for reactants and products. Such foam monolith supports may have 20 to 80, preferably, 30 to 50 pores per inch. Channel monoliths generally have straighter, channel-like pores. These pores are generally smaller, and there may be 80 or more pores per linear inch of catalyst.

Preferred ceramic foams include alumina foams.

Alternatively, the support may be present as a thin layer or wash coat on another substrate.

Where a supported catalyst is employed, the metal components of the catalyst are preferably distributed substantially uniformly throughout the support.

The catalysts employed in the present invention may comprise further elements, such as alkali metals. Suitable alkali metals include lithium, sodium, potassium and cesium.

The catalysts employed in the present invention may be prepared by any method known in the art. For example, gel methods and wet-impregnation techniques may be employed. Typically, the support is impregnated with one or more solutions comprising the metals, dried and then calcined in air. The support may be impregnated in one or more steps. Preferably, multiple impregnation steps are employed. The support is preferably dried and calcined between each impregnation, and then subjected to a final calcination, preferably, in air. The calcined support may then be reduced, for example, by heat treatment in a hydrogen atmosphere.

The catalyst zone may be achieved in any suitable manner provided that the reactant stream (hydrocarbon and oxygen-containing gas) contacts the first catalyst bed thereby producing an effluent stream (comprising reaction products and unreacted feed) therefrom, and said effluent stream passes from the first catalyst bed to the second catalyst bed. A convenient method of achieving the catalyst zone is to use a single reactor with a space being provided between the beds. The space can be provided by placing substantially inert materials such as alumina, silica, or other refractory materials between the catalyst beds.

Alternatively, the space between the catalyst beds is a substantial void.

The space between the catalyst beds is not critical in relation to the beds. Preferably, however, the space will be as small as practical. Most preferably, there is no substantial space between the catalyst beds, that is, the beds are directly adjacent to one another. Where the catalyst zone comprises more than two beds, the size of the space between the beds may vary.

The size of the catalyst beds can vary one from the other. Preferably the size of the first catalyst bed to second catalyst bed is in the ratio of 1:2.

The catalyst beds may be arranged either vertically or horizontally.

The hydrocarbon may be any hydrocarbon which can be converted to an olefin, preferably a mono-olefin, under the partial combustion conditions employed.

The process of the present invention may be used to convert both liquid and gaseous hydrocarbons into olefins. Suitable liquid hydrocarbons include naphtha, gas oils, vacuum gas oils and mixtures thereof. Preferably, however, gaseous hydrocarbons such as ethane, propane, butane and mixtures thereof are employed. Suitably, the hydrocarbon is a paraffin-containing feed comprising hydrocarbons having at least two carbon atoms.

The hydrocarbon feed is mixed with any suitable oxygen-containing gas. Suitably, the oxygen-containing gas is molecular oxygen, air, and/or mixtures thereof. The oxygen-containing gas may be mixed with an inert gas such as nitrogen or argon.

Additional feed components may be included, if so desired. Suitably, methane, hydrogen, carbon monoxide, carbon dioxide or steam may be co-fed into the reactant stream.

Any molar ratio of hydrocarbon to oxygen-containing gas is suitable provided the desired olefin is produced in the process of the present invention. The preferred stoichiometric ratio of hydrocarbon to oxygen-containing gas is 5 to 16, preferably, 5 to 13.5 times, preferably, 6 to 10 times the stoichiometric ratio of hydrocarbon to oxygen-containing gas required for complete combustion of the hydrocarbon to carbon dioxide and water.

The hydrocarbon is passed over the catalyst at a gas hourly space velocity of greater than 10,000 h⁻¹, preferably above 20,000 h⁻¹ and most preferably, greater than 100,000 h⁻¹. It will be understood, however, that the optimum gas hourly space velocity will depend upon the pressure and nature of the feed composition.

Preferably, hydrogen is co-fed with the hydrocarbon and oxygen-containing gas into the reaction zone. The molar ratio of hydrogen to oxygen-containing gas can vary over any operable range provided that the desired olefin product is produced. Suitably, the molar ratio of hydrogen to oxygen-containing gas is in the range 0.2 to 4, preferably, in the range 1 to 3.

Hydrogen co-feeds are advantageous because, in the presence of the catalyst, the hydrogen combusts preferentially relative to the hydrocarbon, thereby increasing the olefin selectivity of the overall process.

Preferably, the reactant mixture of hydrocarbon and oxygen-containing gas (and optionally hydrogen co-feed) is preheated prior to contact with the catalyst. Generally, the reactant mixture is preheated to temperatures below the autoignition temperature of the reactant mixture.

Advantageously, a heat exchanger may be employed to preheat the reactant mixture prior to contact with the catalyst. The use of a heat exchanger may allow the reactant mixture to be heated to high preheat temperatures such as temperatures at or above the autoignition temperature of the reactant mixture. The use of high pre-heat temperatures is beneficial in that less oxygen reactant is required which leads to economic savings. Additionally, the use of high preheat temperatures can result in improved selectivity to olefin product. It has also be found that the use of high preheat temperatures enhances the stability of the reaction within the catalyst thereby leading to higher sustainable superficial feed velocities.

It should be understood that the autoignition temperature of a reactant mixture is dependent on pressure as well as the feed composition: it is not an absolute value. Typically, in auto-thermal cracking processes, where the hydrocarbon is ethane at a pressure of 2 atmospheres, a preheat temperature of up to 450° C. may be used.

The process of the present invention may suitably be carried out at a catalyst exit temperature in the range 600° C. to 1200° C., preferably, in the range 850° C. to 1050° C. and, most preferably, in the range 900° C. to 1000° C.

The process of the present invention may be operated at any suitable pressure, such as at atmospheric pressure or at elevated pressure. The process of the present invention may be operated at a pressure in the range atmospheric to 5 barg, but is preferably operated at a pressure of greater than 5 barg. More preferably the autothermal cracking process is operated at a pressure of between 5-40 barg and advantageously between 10-30 barg e.g. 15-25 barg.

The reaction products are preferably quenched as they emerge from the reaction chamber to avoid further reactions taking place. Usually the product stream is cooled to between 750-600° C. within less than 100 milliseconds of formation, preferably within 50 milliseconds of formation and most preferably within 20 milliseconds of formation e.g. within 10 milliseconds of formation.

Wherein the autothermal cracking process is operated at a pressure of 5-20 barg usually the products are quenched and the temperature cooled to between 750-600° C. within 20 milliseconds of formation. Advantageously wherein the autothermal cracking process is operated at a pressure of greater than 20 barg the products are quenched and the temperature cooled to between 750-600° C. within 10 milliseconds of formation.

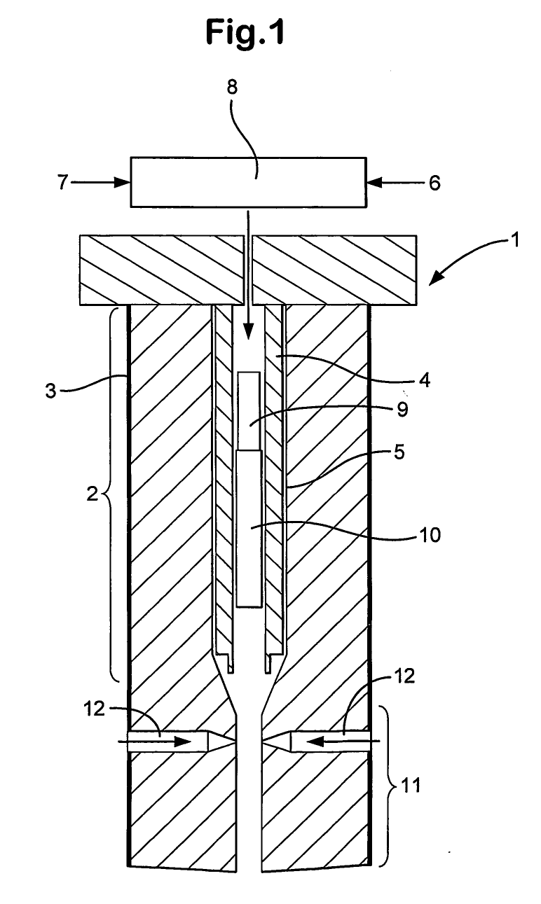

The invention will now be described with the reference to FIG. 1.

FIG. 1 shows a high pressure autothermal reactor (1) a reaction zone (2) surrounded by a pressure jacket (3). The reactor consists of a quartz tubular liner (4) located within a metal holder (5).

Oxygen via line (6) and hydrocarbon feed via line (7) is passed to a gas mixing zone (8). The mixed gaseous reactants are then passed to the reaction zone. The reaction zone comprises a first catalyst bed (9) and a second catalyst bed (10).

As the reactants contact the catalyst beds (9) and (10) some of the hydrocarbon feed combusts to produce water and carbon oxides. This combustion reaction is exothermic and the heat produced is used to drive the dehydrogenation of hydrocarbon feed to a product stream comprising olefins.

The gaseous product stream from the reaction zone passes into a quench zone (11) comprising a gas injection zone (12) wherein it is contacted with a high velocity nitrogen stream at 25° C. to rapidly reduce its temperature and maintain the olefin selectivity.

The invention will now be illustrated in the following examples.

Catalyst Preparation

Catalysts 1 to 3: 3 wt % Platinum on Aluminas

Catalyst 1: 3 wt % Platinum on Alumina Foam

Alumina foam blocks (supplied by Hi-Tech Ceramics, New York, with a porosity of 45 pores per inch (ppi)) were repeatedly impregnated with an aqueous solution of tetrammineplatinum(II), chloride. The tetrammineplatinum(II) chloride solution was prepared with sufficient salt to achieve a nominal Pt loading of 3 wt % if all the metal in the salt were incorporated into the final catalyst formulation. Between impregnations excess solution was removed from the foam blocks, the foam blocks were dried in air at ca. 120° C. for approximately 30 minutes, and subsequently calcined in air at 450° C. for approximately 30 minutes (to decompose the Pt salt to Pt metal on the foam surface). Once all the solution had been absorbed onto the foams the blocks were dried and given a final air calcination at 1200° C. for 6 hours.

Catalyst 2: 3 wt % Platinum on Alumina Spheres

The method of preparation of Catalyst 1 was repeated using alumina spheres (supplied by Condea, 1.8 mm diameter, surface area 210 m²/g) as the support.

It was noted that after calcination the diameter of the spheres had reduced to approximately 1.2 mm.

Catalyst 3: 3 wt % Platinum on Alumina Foam

The method of preparation of Catalyst 1 was repeated using alumina foam blocks with a porosity of 30 ppi (supplied by Hi-Tech, New York) as the support.

Catalysts 4-6: Mixed Metal Oxide Catalysts

Catalyst 4: Y—Ba—Cu Oxide on Alumina

2.084 g of yttrium nitrate hexahydrate (99.9% ex Aldrich), 2.835 g of barium nitrate (99+% ex Aldrich) and 3.975 g of copper(II) nitrate hemipentahydrate (99.99+% ex Aldrich) were dissolved in 50 cm³ of de-ionised water.

Alumina spheres (supplied by Condea, 1.8 mm diameter, surface area 210 m²/g) were repeatedly impregnated with this solution. Between impregnations excess solution was removed from the spheres, the spheres were dried in air at about 120° C. for 10 minutes, and subsequently calcined in air at 450° C. for approximately 30 minutes. Once all the solution had been absorbed onto the spheres they were dried and given a final air calcination at 1200° C. for 6 hours.

It was noted that after calcination the diameter of the spheres had reduced to approximately 1.2 mm.

Catalyst 5: Y—Ba—Cu Oxide

3.65 g of yttrium nitrate hexahydrate (99.9% ex Aldrich), 5.22 g of barium nitrate (99+% ex Aldrich) and 7.248 g of copper(II) nitrate hemipentahydrate (99.99+% ex Aldrich) were mixed thoroughly and placed on a silica tray in an oven at 150° C. for 2 hours. During this time, dissolution and mixing of the salts in their waters of crystallization occurred.

The mixture was then calcined in air at 350° C. for 1 hour, then ramped at 10° C./min to 950° C., where it was held for 4 hours before being cooled.

The resulting solid material was crushed using a mortar and pestle, pressed as 22 mm diameter discs under 20 tonne pressure, then crushed and sieved to 1-2 mm particles.

Catalyst 6: F-doped Y—Ba—Cu Oxide

11.584 g yttrium nitrate hexahydrate (99.9% ex Aldrich), 15.806 g barium nitrate (99+% ex Aldrich), 21.338 g of copper(II) nitrate hemipentahydrate (99.99+% ex Aldrich) and 0.245 g copper fluoride (ex Aldrich, 99.999%) were mixed thoroughly and placed on a silica tray in a drying oven at 350° C. for 3 hours.

The resulting solid material was ground using a mortar and pestle then was calcined in air at 950° C. for 6 hours. The mixture was then allowed to cool, before being re-ground to a powder using a mortar and pestle. This powder was then pressed as 22 mm diameter discs under 20 tonne pressure, then crushed and sieved to 1-2 mm particles and finally re-calcined in air at 950° C. for 6 hours prior to testing

### EXAMPLE 1

A high pressure autothermal reactor as shown in FIG. 1, comprising a first catalyst bed comprising alumina loaded with 3% by weight of platinum (Catalyst 1) was maintained at a pressure of 10 barg. The first catalyst bed had a depth of 30 mm. Ethane, oxygen, hydrogen and nitrogen was passed to the autothermal reactor and the reaction conditions were manipulated such that the ethane conversion was maintained at 50%. The resultant product stream was monitored and its composition is shown in table 1.

The example was repeated using an autothermal reactor comprising a first catalyst bed comprising alumina loaded with 3% by weight of platinum (Catalyst 2) and a second catalyst bed comprising yttrium-barium-copper mixed oxide (Catalyst 4). The second catalyst bed had a depth of 60 mm. The resultant product stream was also monitored and its composition is shown in table 1.

Table 1 shows that the selectivity to ethylene is increased, the selectivity to carbon monoxide is decreased and the oxygen conversion is increased when a second catalyst bed is used in combination with a first catalyst bed.

### EXAMPLE 2

Example 1 was repeated using a pressure of 20 barg and the catalysts listed in Table 2. The results are shown in table 2. It can be seen that again the selectivity to ethylene is increased and the selectivity to carbon monoxide is decreased when a second catalyst bed is used in combination with a first catalyst bed.

### EXAMPLE 3

This example was performed at atmospheric pressure (0 barg) in an autothermal reactor comprising a quartz reactor in an electrically heated furnace, as described in WO 02/04389. With a first catalyst bed comprising alumina loaded with 3% by weight of platinum (Catalyst 3), ethane, oxygen, hydrogen and nitrogen was passed to the autothermal reactor and the reaction conditions were manipulated such that the ethane conversion was maintained at ca. 40%. The resultant product stream was monitored and its composition is shown in table 3.

The example was repeated using a first catalyst bed comprising alumina loaded with 3% by weight of platinum (Catalyst 3) and a second catalyst bed comprising yttrium-barium-copper mixed oxide (Catalyst 5). The resultant product stream was also monitored and its composition is shown in table 3.

It can be seen that again the selectivity to ethylene is increased and the selectivity to carbon monoxide is decreased when a second catalyst bed is used in combination with a first catalyst bed.

### EXAMPLE 4

Example 3 was repeated but using a second catalyst bed comprising fluoride-doped yttrium-barium-copper mixed oxide (Catalyst 6). The results are shown in table 4. It can be seen that again the selectivity to ethylene is increased and the selectivity to carbon monoxide is decreased when a second catalyst bed is used in combination with a first catalyst bed.

TABLE 1
Ethane autothermal cracking at 10barg with hydrogen
co-feed, at ca. 50% ethane conversion

| Catalyst bed 1 | | Catalyst 1 | Catalyst 2 |
|---|---|---|---|
| Catalyst bed 2 | | — | Catalyst 4 |
| Feed rates | | | |
| Ethane | g/min | 101.20 | 99.85 |
| Hydrogen | g/min | 2.44 | 2.40 |
| Oxygen | g/min | 37.05 | 35.89 |
| nitrogen to reactor | g/min | 11.08 | 11.10 |
| quench nitrogen | | 147.97 | 50.06 |
| Pressure | barg | 10.02 | 9.99 |
| feed preheat | ° C. | 158 | 182 |
| Catalyst | ° C. | 898 | 898 |
| Post Nitrogen-Quench | ° C. | −495 | 520 |
| ethane conversion | % | 48.09 | 50.20 |
| oxygen conversion | % | 90.82 | 99.65 |
| Selectivity (g per 100 g | | | |
| ethane converted) | | | |
| Hydrogen | | 5.49 | 5.03 |
| Methane | | 10.25 | 9.01 |
| Ethylene | | 61.09 | 67.59 |
| Acetylene | | 0.22 | 0.26 |
| Propane | | 1.74 | 0.91 |
| Propylene | | 3.60 | 2.84 |
| Butane | | 2.91 | 1.34 |
| Butenes | | 0.66 | 0.52 |
| Butadiene | | 0.76 | 0.94 |
| C5-C7 | | 0.57 | 0.17 |
| Aromatics | | 0.16 | 0.11 |
| carbon monoxide | | 22.71 | 13.76 |
| carbon dioxide | | 4.44 | 12.31 |

TABLE 2
Ethane autothermal cracking at 20barg with hydrogen
co-feed, at ca. 50% ethane conversion

| catalyst bed 1 | | Catalyst 2 | Catalyst 2 |
|---|---|---|---|
| catalyst bed 2 | | — | Catalyst 4 |
| feed rates | | | |
| Ethane | g/min | 199.87 | 198.06 |
| Hydrogen | g/min | 4.48 | 5.18 |
| Oxygen | g/min | 71.68 | 71.95 |
| Nitrogen | g/min | 11.12 | 11.09 |
| pressure | barg | 20.02 | 19.93 |
| feed preheat | ° C. | 162 | 156 |
| catalyst temp #1 | ° C. | 909 | 903 |
| post nitrogen quench | ° C. | 696 | 527 |
| ethane conversion | % | 49.90 | 50.94 |
| oxygen conversion | % | 99.65 | 99.89 |
| Selectivity (g per 100 g | | | |
| ethane converted) | | | |
| Methane | | 11.17 | 12.08 |
| CO | | 21.44 | 18.96 |
| CO2 | | 4.08 | 7.05 |
| Ethylene | | 56.17 | 58.29 |
| Acetylene | | 0.18 | 0.26 |
| Propylene | | 4.60 | 4.61 |
| Propane | | 1.42 | 1.22 |
| MAPD | | 0.05 | 0.00 |
| Butane | | 3.06 | 1.93 |
| Butenes | | 1.36 | 1.17 |
| Butadiene | | 1.44 | 1.28 |
| C5-C7 | | 1.90 | 1.19 |
| Aromatics | | 1.35 | 1.06 |

TABLE 3
Ethane autothermal cracking at 0barg with hydrogen
co-feed, at ca. 40% ethane conversion

| Catalyst bed 1 | | | Catalyst 3 |
|---|---|---|---|
| Catalyst bed 2 | | Catalyst 3 | Catalyst 5 |
| Feed rates | | | |
| Ethane | g/min | 4.98 | 4.98 |
| Hydrogen | g/min | 0.20 | 0.20 |
| Oxygen | g/min | 1.60 | 1.60 |
| Nitrogen | g/min | 1.03 | 0.98 |
| Pressure | Barg | 0.00 | 0.00 |
| feed preheat | ° C. | 348 | 323 |
| Catalyst | ° C. | 823 | 825 |
| ethane conversion | % | 41.6 | 44.7 |
| oxygen conversion | % | −98.4 | 100.0 |
| Selectivity (g per 100 g | | | |
| ethane converted) | | | |
| Methane | | 6.11 | 4.35 |
| Ethylene | | 71.34 | 75.77 |
| Acetylene | | 0.00 | 0.00 |
| Propane | | 1.04 | 0.39 |
| Propylene | | 1.27 | 0.75 |
| Butane | | 1.95 | 0.57 |
| Butenes | | 0.17 | 0.14 |
| Butadiene | | 0.23 | 0.74 |
| C5-C7 | | 0.05 | 0.02 |
| Aromatics | | 0.00 | 0.00 |
| carbon monoxide | | 17.02 | 12.53 |
| carbon dioxide | | 7.59 | 12.09 |

TABLE 4
Ethane autothermal cracking at 0barg with hydrogen
co-feed, at ca. 40% ethane conversion

| Catalyst bed 1 | | Catalyst 3 | Catalyst 3 |
|---|---|---|---|
| Catalyst bed 2 | | — | Catalyst 6 |
| Feed rates | | | |
| Ethane | g/min | 4.46 | 4.46 |
| Hydrogen | g/min | 0.22 | 0.22 |
| Oxygen | g/min | 1.78 | 1.78 |
| Nitrogen | g/min | 1.02 | 0.99 |
| Pressure | Barg | 0.00 | 0.00 |
| feed preheat | ° C. | 219 | 229 |
| Catalyst | ° C. | 827 | 887 |
| ethane conversion | % | 41.1 | 41.4 |
| oxygen conversion | % | −94.7 | 100.0 |
| Selectivity (g per 100 g | | | |
| ethane converted) | | | |
| Methane | | 5.77 | 3.49 |
| Ethylene | | 72.70 | 76.65 |
| Acetylene | | 0.00 | 0.00 |
| Propane | | 1.22 | 0.35 |
| Propylene | | 1.55 | 0.69 |
| Butane | | 2.41 | 0.75 |
| Butenes | | 0.40 | 0.19 |
| Butadiene | | 0.07 | 0.41 |
| C5-C7 | | 0.02 | 0.12 |
| Aromatics | | 0.00 | 0.00 |
| carbon monoxide | | 15.26 | 11.19 |
| carbon dioxide | | 4.07 | 13.92 |

1-9. (canceled)

10. A process for the production of an olefin, said process comprising passing a mixture of a hydrocarbon and an oxygen-containing gas through a catalyst zone comprising at least a first catalyst bed and a second catalyst bed, wherein the first catalyst bed comprises a catalyst which is capable of supporting combustion beyond the fuel rich limit of flammability and wherein the second catalyst bed is located downstream of the first catalyst bed, is of a different composition to the first catalyst bed, has the general formula of:
M¹ₐM²_{b}M³_{c}O_{z}
wherein M¹ is selected from groups IIA, IIB, IIIB, IVB, VB, VIIB, VIIB, lanthanides and actinides, M² is selected from groups IIA, IB, IIB, IIIB, IVB, VB, VIB, M³ is selected from groups IIA, IB, IIB, IIIB, IVB, VB, VIB and VIIIB, a, b, c and z are the atomic ratios of components M¹, M², M³ and 0 respectively, a is in the range of 0.1 to 1.0, b is in the range of 0.1 to 2.0, c is in the range of 0.1-3.0, and z is in the range 0.1 to 9, and has a perovskite-type structure.

11. A process according to claim 10 wherein the first catalyst bed comprises a Group VIIIB metal.

12. A process according to claim 11 wherein the first catalyst bed is selected from the group consisting of Pt/Ga, Pt/In, Pt/Sn, Pt/Ge, Pt/Cu, Pd/Sn, Pd/Ge, Pd/Cu and Rh/Sn.

13. A process according to claim 10 wherein M¹ is selected from group IIIB, M² is selected from group IIA and M³ is selected from group IB.

14. A process according to claim 13 wherein M¹ is yttrium, M² is barium and M³ is copper.

15. A process according to claim 10 wherein the second catalyst bed is promoted by addition of halide-promoters to yield materials having the general formula of;
M¹ₐM²_{b}M³_{c}XₓO_{z}
wherein X is a halide and x is in the range of 0.05-0.5.

16. A process according to claim 10 wherein the hydrocarbon is a paraffin-containing feed comprising hydrocarbons having at least two carbon atoms.

17. A process according to claim 10 wherein the molar ratio of hydrocarbon to the oxygen-containing gas is 5 to 16 times the stoichiometric ratio of hydrocarbon to oxygen-containing gas required for complete combustion of the hydrocarbon to carbon dioxide and water.

18. A process according to claim 10 wherein hydrogen is co-fed with the hydrocarbon and oxygen-containing gas into the reaction zone.
